(19) United States
(12) Patent Application Publication
(10) Pub. No.: US 2017/0265736 A1
(43) Pub. Date: Sep. 21, 2017

(54) BIFLOW SPINAL CANNULA

(71) Applicants: James K. Brannon, Leawood, KS (US); Kristen Radcliff, Longport, NJ (US)

(72) Inventors: James K. Brannon, Leawood, KS (US); Kristen Radcliff, Longport, NJ (US)

(21) Appl. No.: 15/074,732

(22) Filed: Mar. 18, 2016

(51) Int. Cl. A61B 1/317; A61B 17/02; A61B 17/34; A61B 1/32

(52) U.S. Cl. CPC A61B 1/317; A61B 1/32; A61B 17/0218; A61B 17/3423; A61B 17/3415; A61B 2217/007; A61B 2017/3454; A61B 2090/062

(57) ABSTRACT

An improved cannula that provides improved visibility at a surgical site through the continuous flow of fluid through a cannula positioned in direct proximity of the surgical site, the improved cannula including a handle, a pair of portals, and a trephine.

### FIELD OF THE INVENTION

The present invention is broadly directed to improvements in instruments for arthroscopic surgery and, more particularly, to a device which provides improved visibility of the surgical site during arthroscopy on the vertebral pedicle.

### BACKGROUND OF THE INVENTION

Modern surgery tends toward minimally invasive techniques whenever possible because they reduce pain and accelerate healing. Although often more complicated in some ways for the surgeon, minimally invasive techniques result in less trauma to the patient and less scarring because of much smaller incisions thereby promoting faster healing and reducing possibilities for infections. In general, minimally invasive surgeries involve making one or more small incisions at appropriate locations and inserting tubular devices through the incisions to the surgical site. Arthroscopic surgery typically requires visibility of the surgical site, and in many cases, it is necessary to inspect the surgical site prior to performing a procedure. During the procedures, byproducts from use of instruments, debris, and blood can enter the space around the surgical site and cause poor visual clarity.

Depending on the procedures, different techniques are relied upon to maintain good visibility. To help improve visibility, some surgeons rely upon the cannulae or the effective removal of debris. However, when there is limited room to maneuver within a surgical site, and because of the close proximity, it is desirable to maintain accessibility while limiting any visual obstructions from the surgical site. One feature of good visibility is the effective removal of surrounding debris. Another is that the user has an unobstructed view of the instrument in contact with the tissue and the view of the tissue not be obscured by the instrument.

In a typical procedure, removal of debris from the visual field is accomplished by aspirating debris from the surgical site via an inner rotating lumen, which is connected to an external vacuum source. However, in some procedures the surgical site is too delicate and small for surgical tools that aspirate debris and remove that debris through an external vacuum source. Moreover, many procedures create little debris, and the use of an external vacuum source is unnecessary. When there is minimal debris and the visual field is primarily obstructed by blood flow, devices relying on external suction are ineffective. Such procedures would require different tools and different methods for maintaining visibility of the surgical site than those currently known in the art. As such, there is currently a problem with maintaining visually clarity in a small and delicate surgical site.

There is a need for an improved cannula, which provides for improved visibility during surgery while not obstructing contact with the tissue. In addition, there is a need for an improved cannula with characteristics to prevent and remove blood from entering the surgical site, which enhances the surgeon's visibility in procedures where visibility is required.

The current invention addresses this problem by visual clarity of a surgical site with the use of the present invention. In one embodiment, the current invention provides a continual flow of fluid to a surgical site, allowing for removal of visually obstructing debris and blood from the surgical site and providing for improved visual clarity at the surgical site.

### SUMMARY OF THE INVENTION

The present invention is a cannula that provides improved visibility at a surgical site through the continuous flow of fluid through a cannula positioned in direct proximity of the surgical site. The invention includes a handle, a pair of portals, and a trephine.

An embodiment of the invention includes a handle communicating with a trephine terminating distally therefrom. The handle will remain external to the patient, while the trephine will be inserted through an incision toward the surgical site. A side portal and a rear portal, respectively, communicate with the handle. Generally, the rear portal is aligned with the trephine but spaced from the trephine by the handle. Internally, the handle contains a central mixing chamber that allows for simultaneous fluidic communication between the side portal, the rear portal, and the surgical site through the trephine. The trephine is configured to establish and maintain a hermetic seal with a membrane of the surgical site such as a vertebral pedicle.

The trephine includes a conical trephine body terminating distally in a trephine tip, which may be conically tapered. The trephine tip contains a plurality of flow apertures adaptable for the inflow or outflow of fluid at the surgical site. In an alternative embodiment the trephine tip includes a plurality of elongated slots which are adapted for the inflow and outflow of fluid. The trephine has an internal trephine mixing chamber with a diameter sufficient for receipt of endoscopic instruments while is passed simultaneously therethrough. The trephine may be constructed from any suitable material, such as stainless steel, a polymer, or the like.

The present invention is used to provide visibility to a surgical site through the controlled continual flow of fluid, while endoscopic instruments are extended through the trephine chamber and beyond the trephine tip into the surgical site. The trephine is inserted through an incision and forms a hermetic seal with a membrane or barrier to the surgical site. In operation, an endoscopic instrument may be inserted through the rear portal and passed through the trephine chamber for placement along the trephine. A pressurized fluid source may be secured to the side portal of the handle. The fluid from the fluid source enters through the side portal of the handle, travels through the internal mixing chamber to the trephine and exits through the trephine tip into the surgical site providing improved visibility. The pressurized fluid source may include an irrigant such as water, a saline solution, or other solution of a type that would be used to irrigate a surgical site. The fluid source generally provides a positive pressure to the surgical site, the fluid source presenting a pressure greater than the patient's blood pressure which may range between 60 and 160 mm of Hg.

In an alternative embodiment, a trocar or other endoscopic surgical instrument is received by the rear portal of the handle and extended through the trephine chamber beyond the trephine tip. The trocar having a rear cap and trocar body extending distally therefrom with a length greater than the cannula. The rear cap is capable of communicating with the rear of the handle to form a lock, which holds the trocar in place. The trocar being adapted for extension through the trephine chamber and beyond the trephine tip into the surgical site.

In an exemplary embodiment of the invention, the trephine is inserted into a patient's pedicle with the outer surface at the distal end of the trephine making a hermetic seal with the proximal end of the patient's pedicle. The trocar is then received by the rear port of the handle and inserted through the trephine. The trocar cannulates the pedicle and in doing so, compresses cancellous bone tissue within the pedicle centrifugally thereby reducing blood flow from the internal surface area of the pedicle. The fluid source is connected to the side portal of the handle and fluid travels into the internal area of the patient's pedicle. The hermetic seal prevents fluid from escaping and keeps the pressure of the fluid higher than the patient's blood pressure. An internal endoscope is then positioned along the internal surface of the trephine towards the surgical site in such a manner that still allows for fluid to flow in the same direction. The fluid continues to flow into the internal area of the patient's pedicle when the internal endoscope is moved beyond the trephine tip and allows for a clear view of the internal bony surface of the patient's pedicle. The surgeon may then visually inspect the internal area of the patient's pedicle.

Additionally, the trocar is slightly larger in diameter than the endoscope, which allows the endoscope to be telescoped through the path created by the trocar in the pedicle.

Various objects and advantages of the present invention will become apparent from the following description taken in conjunction with the accompanying drawings wherein are set forth, by way of illustration and example, certain embodiments of this invention. The drawings submitted herewith constitute a part of this specification, include exemplary embodiments of the present invention, and illustrate various objects and features thereof.

### BRIEF DESCRIPTION OF THE DRAWINGS

FIG. 1 is a top elevation view of an embodiment of the present invention.

FIG. 2 is a side elevation view of FIG. 1.

FIG. 3 is a cross-sectional view taken on line A of FIG. 2.

FIG. 4 is a side elevation view of an embodiment of the present invention with a surgical instrument.

FIG. 5. is a side elevation view of an alternative embodiment of the present invention.

FIG. 6 is a side elevation view of the cap.

FIG. 7 is a side elevation view of the trocar.

FIG. 8. is a side elevation view of the trocar with an alternative tip.

FIG. 9. is a side elevation view of the trocar with an alternative tip.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 10 generally designates an embodiment of the biflow spinal cannula instrument (hereinafter “cannula”). The cannula includes a handle 20 with a trephine 30 extending distally therefrom.

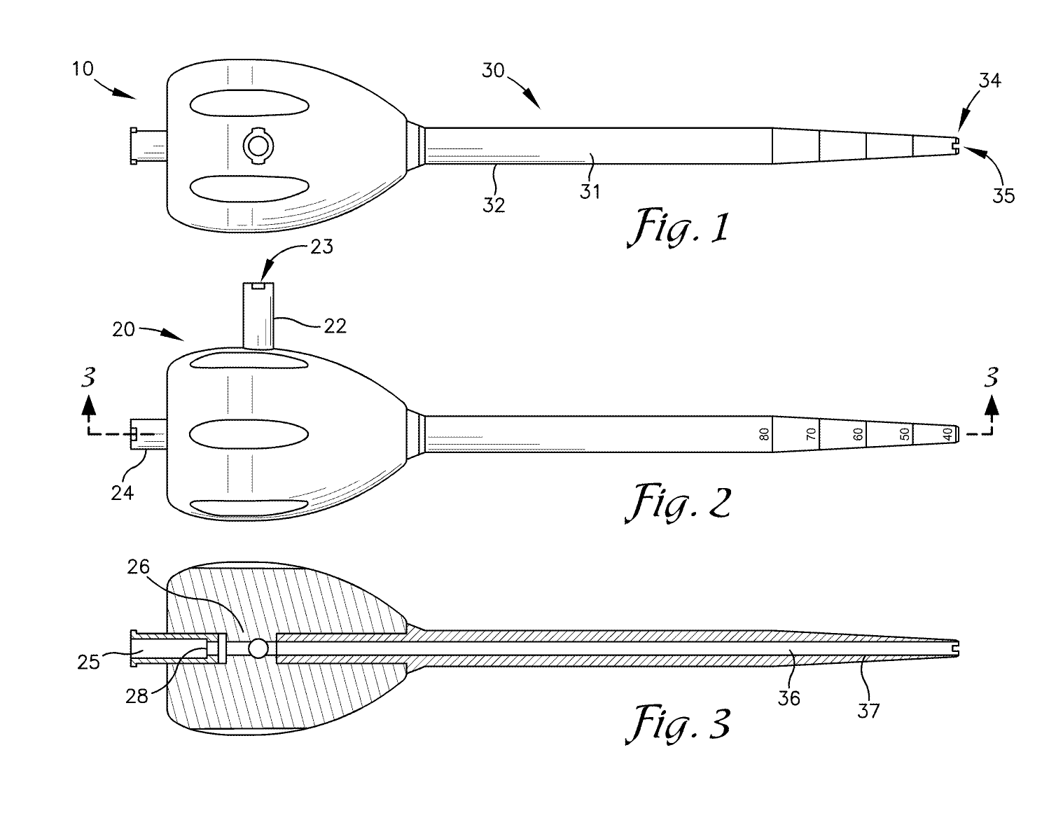

As depicted in FIGS. 1-3, an embodiment of the cannula 10 includes a handle 20 that contains a central mixing chamber 26. The handle 20 is acorn-shaped with a plurality contoured elongations along the surface for grip. A side portal 22 radially extends outward from the handle and is adapted for receipt of a fluid source. A first chamber 23 extends inwardly from the side portal 22 through the handle 20 to the central mixing chamber 26 for fluidic communication therebetween. A rear portal 24 extends proximately from the rear of the handle 20 and is adapted for the receipt of other endoscopic instruments, including a trocar 40 or visual instrument. A second chamber 25 that extends inwardly from the rear portal 24 through the handle 20 to the central mixing chamber 26 for communication therebetween.

The rear portal chamber 25 may also include an o-ring 28 presenting a hermetic seal limiting fluid flow through the rear portal while permitting removable receipt of a plurality of various surgical instruments, including but not limited to a trocar or optical instrument received at the rear portal 24.

A trephine 30 extends from the handle 20 with the trephine 30 being axially aligned with the rear portal 24. Both the side portal 22 and the rear portal 24 may include repeatable releasable locking mechanisms (not shown) to hold fluid sources and/or instruments received by the cannula 10 in place during use. One such example may include but is not limited to a leur lock.

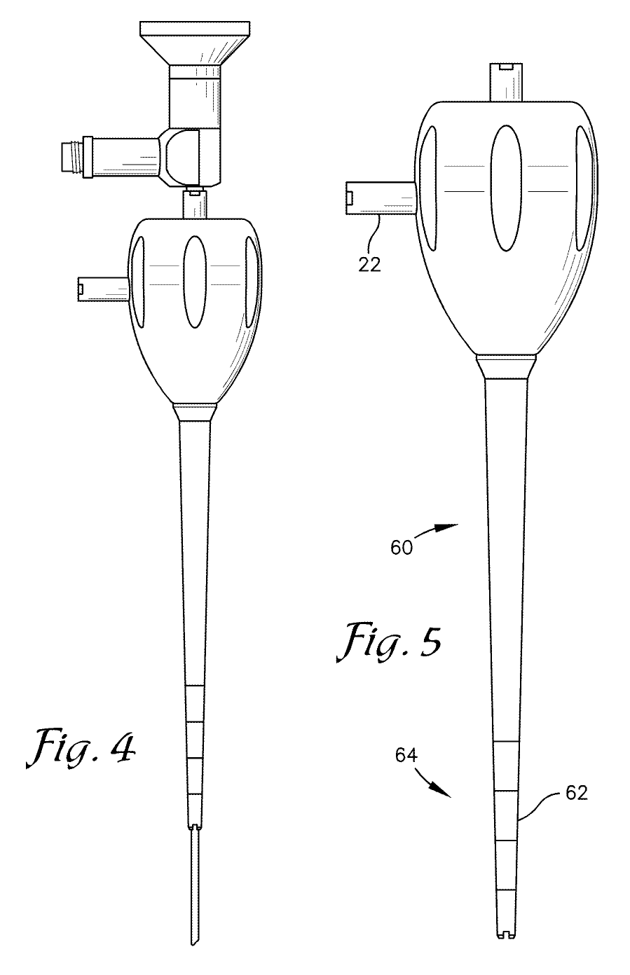

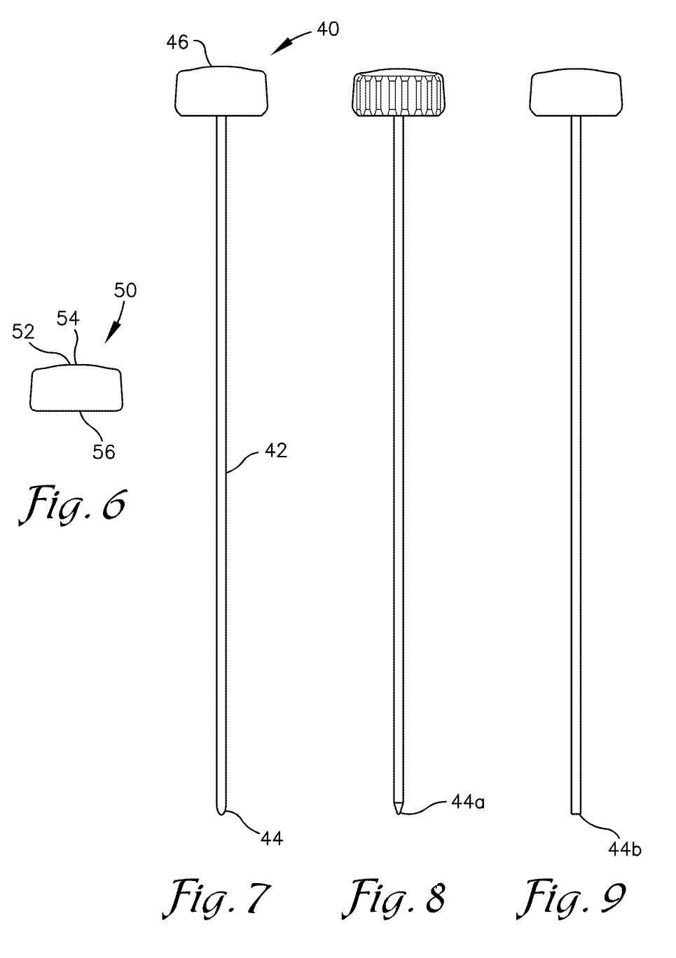

In one embodiment of the cannula 10, the trephine 30 extends forwardly from the handle 20 and includes a conical trephine body 32, which terminates distally at a trephine tip 34. The proximal end of trephine body 32 has a barbed flange which is adjacent to the handle 20. As illustrated in FIGS. 1-5, a plurality of flow apertures 35 also referred to as sidewall apertures are located on opposite sides of the trephine top 34 and are adapted for the inflow and outflow of fluid as desired during surgical procedure where the trephine tip may be obstructed with, for example, a surgical instrument or surrounding tissue or bone.

Alternative flow apertures (not shown) may be positioned along the outer surface including a plurality of elongated slots or apertures (not shown) being positioned near the trephine tip 34 and further adapted for the inflow and outflow of fluid from the surgical site.

The trephine 30 illustrated in FIGS. 1-3 contains a chamber 36 that extends continuously with a constant radius from the central mixing chamber 26 and through the trephine body 32 terminating at the trephine tip 34. The trephine 30 contains an outer surface 32 and the trephine chamber 36 contains an inner surface 37, the outer surface and inner surface being radially separated by a sidewall thickness, which generally corresponds to the thickness of the trephine 30 and may vary according to the desired fabrication methods and desired operational characteristics. The trephine chamber 36 is in fluidic communications with the central mixing chamber 26 and is adapted for simultaneous receipt of fluid from the fluid source at the side portal 22 and surgical instruments received from the rear portal 24.

As illustrated in FIG. 5, an alternative trephine 60 may include a plurality of vertically spaced indicia 62, the indicia circumscribing the trephine 60, with each indicia extending along the trephine outer surface near the distal end of the trephine 64. Each indicia corresponds to a given location. By way of example, the indicia illustrated in FIG. 5 are positioned coaxial to the side portal 22 and generally allow for localization of fractures or other pathology associated with the surgical site.

The central mixing chamber 26 simultaneously communicates with each of the side portal chamber 23, the rear portal chamber 25, and the trephine chamber 36, independently. In this manner, the trephine chamber 36 can simultaneously communicate with the side portal 23 and the rear portal 25. Thus, the trephine chamber 36 can simultaneously and continuously receive fluid from the fluid source at the side portal chamber 23 and surgical instruments from the rear portal chamber 25.

In an alternative embodiment, a trocar 40 can be inserted into the rear portal 24 through the central mixing chamber 26 and the trephine chamber 36 and into the surgical site. The trocar has body 42, a tip 44 at the distal end, and a top 46 at the proximal end. The tip 44 may have multiple embodiments as shown in FIGS. 7-9. The tip may be sharp 44a or blunt 44b based upon the desired operational characteristics.

In an additional embodiment, a cap 50 may be placed on the rear portal 24 in order to shorten the working length of surgical instruments received by the rear portal 24. The cap 50 contains an aperture 52 adapted for receipt of surgical instruments including the trocar 40. The cap 50 may also include an o-ring 54 adapted for creating a hermetic seal with received surgical instruments. Further, the cap contains a locking mechanism 56, such as but not limited to a leur lock so that it may be held in place when adapted to the rear portal 24.

It is to be understood that while certain forms of the present invention have been illustrated and described herein, it is not to be limited to the specific forms or arrangement of parts described and shown.

1. A bidirectional flow surgical cannula apparatus for providing simultaneous and continuous fluid flow and surgical instrument access to the surgical site during arthroscopy, the apparatus comprising:
an elongated trephine with a barbed flange Are located on opposite sides of the trephine tip 34
at the proximal end separated from a trephine tip at the distal end by a conical body;
said trephine tip including a plurality of sidewall apertures adapted for simultaneous fluidic communication with the surgical site;
said proximal end of the elongated trephine attached to an acorn-shaped handle containing a plurality of contoured elevations on its surface and an internal central mixing chamber adapted for fluidic communication with the surgical site;
said elongated trephine containing an internal trephine chamber with a constant radius that extends continuously from said central mixing chamber to said trephine tip and adapted for fluidic communication between the surgical site and said central mixing chamber;
said handle presenting a rear portal being axially aligned with said internal trephine chamber and adapted for receipt of a surgical instrument;
said handle presenting a side portal radially extending outward from said central mixing chamber;
a first chamber extending inwardly from said side portal through said handle to said central mixing chamber adapted for fluidic communication between the surgical site and said side portal; and
a second chamber extending inwardly from said rear portal through said handle to the central mixing chamber adapted for communication between the surgical site and said rear portal.

2. The apparatus as recited in claim 1, wherein said elongated trephine contains a plurality of vertically spaced indicia extending around the circumference of said elongated trephine near the distal end of said elongated trephine.

3. The apparatus as recited in claim 1, wherein:
said internal trephine chamber is adapted for receiving a trocar through said rear portal; and
said trocar having a substantially cylindrical top at the proximal end separated from a trocar tip at the distal end by a rigid cylindrical trocar body.

4. The apparatus as recited in claim 1, further comprising a cap secured to said rear portal containing a cap aperture adapted for receipt of a surgical instrument.
